(19) United States
(12) Patent Application Publication
(10) Pub. No.: US 2019/0183464 A1
(43) Pub. Date: Jun. 20, 2019

(54) METHOD FOR USE IN PERFORMING A PROSTATECTOMY

(71) Applicant: Henry Ford Health System, Detroit, MI (US)

(72) Inventor: Mani Menon, Bloomfield Hills, MI (US)

(21) Appl. No.: 16/223,991

(22) Filed: Dec. 18, 2018

Related U.S. Application Data
(60) Provisional application No. 62/599,814, filed on Dec. 18, 2017.

(51) Int. Cl. A61B 10/02; A61B 17/3205

(52) U.S. Cl. CPC A61B 10/0241; A61B 2017/320064; A61B 17/3205

(57) ABSTRACT

A method for use in performing a prostatectomy procedure on a patient. The method includes: identifying a dominant lesion in a prostate gland, wherein the prostate gland is contained within a prostate capsule; cutting outside the perimeter of a first portion of the prostate capsule corresponding to a region of the prostate gland in which the dominant lesion is located, and inside the perimeter of a second portion of the prostate capsule corresponding to a region of the prostate gland in which the dominant lesion is not located; and removing the first portion of the prostate capsule from the patient's body and preserving the second portion of the prostate capsule in the patient's body.

### CROSS REFERENCE TO RELATED APPLICATIONS

This application which claims the benefit of U.S. Provisional Patent Application No. 62/599,814 filed Dec. 18, 2017, the entire contents of which are hereby incorporated by reference.

### TECHNICAL FIELD

This disclosure relates generally to a method for use in performing a surgical procedure, and more particularly, to a method for use in performing a prostatectomy.

### BACKGROUND

In conventional prostatectomy procedures, the patient's entire prostate, which includes both a prostate capsule and a prostate gland contained within the capsule, is typically removed from the patient's body by cutting outside the entire perimeter of the prostate capsule containing the prostate. This is often referred to as a “radical removal.” While effective to remove the cancer in the prostate, removal of the entire prostate may result in undesirable side effects, including, for example, sexual and/or urinary dysfunction.

More particularly, specialized magnetic resonance imaging (MRI) looking specifically at nerves surrounding the prostate have shown a complicated network of about 1,000-3,000 nerves that surround the prostate. These nerves are intertwined with blood vessels, connective tissue, and muscle fibers. The network starts at the back of the prostate near its junction with the bladder and runs on the side of the prostate turning to the front at the apex of the prostate before entering the penis. At the sides of the apex, the nerves are closely adherent to the prostate capsule. Because conventional prostatectomy procedures involve cutting outside the perimeter of the prostate capsule (i.e., removing the entirety of the capsule), the nerves running into the prostate capsule are removed or peeled back, which may result in at least temporary injury to those nerves.

Through pathological examination of several thousand samples of prostate cancer surgically removed from patients, it was found that most men with prostate cancer have a “dominant” or large lesion in one side of the prostate (e.g., on one side of a midline extending between the anterior and posterior of the prostate), and smaller lesions elsewhere in the gland. Examination has shown that the most aggressive cancer is located in the dominant lesion. As such, in certain instances, removing the entire prostate when the cancer, or at least the most aggressive cancer, is located only in one region of the prostate may subject the patient to side effects that they may not otherwise be subjected to (or at least not to the same degree) if the entire prostate was not removed.

Accordingly, there is a need for a method for use in performing a prostatectomy that minimizes and/or eliminates one or more of the above-identified deficiencies in conventional prostatectomy procedures.

### SUMMARY

According to one embodiment of the invention, there is provided a method for use in performing a prostatectomy procedure on a patient. The method includes: identifying a dominant lesion in a prostate gland, wherein the prostate gland is contained within a prostate capsule; cutting outside the perimeter of a first portion of the prostate capsule corresponding to a region of the prostate gland in which the dominant lesion is located, and inside the perimeter of a second portion of the prostate capsule corresponding to a region of the prostate gland in which the dominant lesion is not located; and removing the first portion of the prostate capsule from the patient's body and preserving the second portion of the prostate capsule in the patient's body.

According to another embodiment of the invention, there is provided a method for use in performing a prostatectomy procedure on a patient. The method includes: identifying a dominant lesion in a prostate gland, wherein the prostate gland is contained within a prostate capsule; collecting one or more samples of the prostate capsule and testing the sample(s) for cancer cells; identifying, based on the testing of the sample(s), a first portion of the prostate capsule to remove from the patient's body, and one or more other portions of the prostate capsule to preserve; cutting outside the perimeter of the first portion of the prostate capsule to be removed, and inside the perimeter of the one or more other portions of the prostate capsule to be preserved; and removing from the patient's body the first portion of the prostate capsule and preserving the one or more other portions of the prostate capsule in the patient's body.

According to yet another embodiment of the invention, there is provided a method for use in performing a prostatectomy procedure on a patient. The method includes: identifying a dominant lesion in a prostate gland, wherein the prostate gland is contained within a prostate capsule; identifying a first portion of the prostate capsule to remove from the patient's body and a second portion of the prostate capsule to preserve; and removing the first portion of the prostate capsule from the patient's body and preserving the second portion of the prostate capsule in the patient's body.

### BRIEF DESCRIPTION OF THE DRAWINGS

One or more embodiments of the invention will hereinafter be described in conjunction with the appended drawings, wherein like designations denote like elements, and wherein:

FIG. 1 is a depiction of an image illustrating, in part, an example of a prostate and the network of nerves surrounding the prostate;

FIG. 2 is a flowchart of an embodiment of a method for use performing a prostatectomy procedure; and

FIG. 3 is a flowchart of another embodiment of a method for use in performing a prostatectomy procedure.

### DETAILED DESCRIPTION

In an illustrative embodiment, a method for use in performing a prostatectomy comprises performing a radical removal (i.e., cutting outside the perimeter of the prostate capsule) on only a portion of the prostate where a dominant lesion is located within the prostate gland (e.g., on the side of the prostate where the dominant lesion is located). For other portions of the prostate containing either no lesions or smaller, lower risk lesions (e.g., the side of the prostate opposite the side containing the dominant lesion), the method further comprises removing that portion of the prostate gland but doing so by cutting within the perimeter of the prostate capsule (e.g., 1-5 mm from the periphery or outer surface of the prostate capsule) so that, for those areas, a rim of tissue that forms part of the prostate capsule is left intact. The part of the capsule or capsular tissue remaining following the cutting steps above is referred to below as “remnant capsular tissue.” Following the cutting described above, the portions of the prostate and prostate capsule containing both the dominant and smaller lesions are removed from the patient's body, while the remnant capsular tissue is preserved (i.e., not removed from the patient's body, but rather left at least substantially intact). It will be appreciated that in an embodiment, all of the cutting described above is performed prior to the removal of any tissue. In other embodiments, however, tissue may be removed as the cutting step(s) is/are performed such that after tissue is removed, additional cutting may be performed.

In one embodiment, after the tissue containing lesions is removed, one or more real-time, intra-operative biopsies may be performed on one or more samples of remnant capsular tissue to test for the presence of cancer cells in the remnant capsular tissue. In other embodiments, one or more samples may be collected from various locations of the prostate capsule after identification of the dominant lesion and/or prior to any cutting of the prostate (i.e., not intra-operatively), and biopsies may be performed on that or those samples to test for the presence of cancer cells in the capsular tissue. Regardless of when the samples are collected and the biopsies performed, any number of samples may be collected and biopsies performed to thoroughly examine the capsular tissue. For example, in an embodiment, 1-15 biopsies are performed, and in one particular embodiment, 8-10 biopsies are performed. It will be appreciated, however, that the present disclosure is not intended to be limited to any particular number of samples/biopsies.

If it is determined from the biopsies that cancer cells are not present at the tested location(s) of the prostate capsule, the tissue at those locations may be preserved. If, however, it is determined that cancer cells are present at one or more locations of the prostate capsule, a further determination may be made that at least the part(s) of the prostate capsule corresponding to that or those locations should be removed. In some instances, this may result in the procedure being effectively converted into a radical prostatectomy in which the entirety of the prostate gland and prostate capsule are removed. In other instances, however, parts of the prostate capsule corresponding to locations determined not to contain cancer cells may be preserved (i.e., not removed and left at least substantially intact).

In an instance where at least some of the prostate capsule tissue is preserved and left substantially intact, some or all of the nerves connected to or surrounding that tissue are also left intact, which provides an added layer of protection to the network of nerves involved and lessens the chance of nerve damage, allowing for the maintenance or earlier return of urinary and/or erectile function.

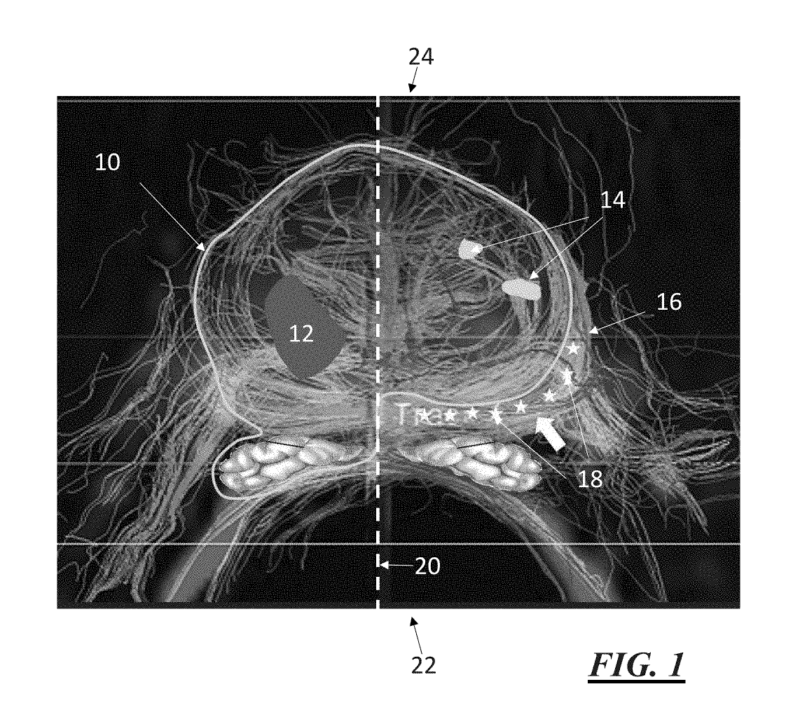

FIG. 1 depicts an image of a prostate and the network of nerves surrounding the prostate. Also depicted is a template 10 representing the area of the prostate that is to be removed during the prostatectomy procedure performed in accordance with one embodiment of the present disclosure described herein, and that includes both a dominant lesion 12 and smaller lesions 14. FIG. 1 also depicts the remnant capsular tissue 16 of the prostate capsule that is preserved after the removal of the cancerous tissue, and possible biopsy sites or locations 18 thereon. The dominant lesion 12 is located on a first side of the prostate with respect to an anterior-posterior midline 20, which extends from the posterior 22 to the anterior 24 of the prostate. The possible biopsy sites or locations 18 are located on a second side (or the other side) of the anterior-posterior midline 20 opposite from the dominant lesion. In at least some embodiments, including the illustrated embodiment, the template 10 may encompass a seminal vesicle and/or vas deferens that are on the same side of the anterior-posterior midline 20 as the dominant lesion 12, which is the left side of the anterior-posterior midline 20 as depicted in FIG. 1. And, in at least some embodiments, a seminal vesicle and/or vas deferens that are on the opposite side of the anterior-posterior midline 20 as the dominant lesion 12 (e.g., the right side in FIG. 1) may be preserved.

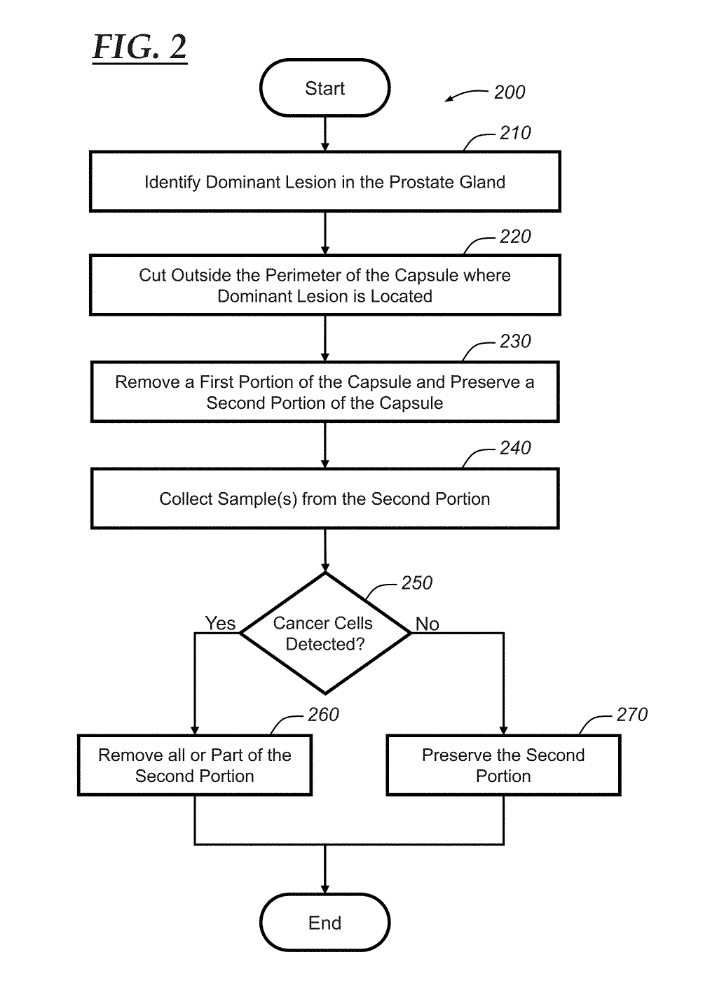

FIG. 2 depicts a flowchart illustrating a first embodiment of a method (method 200) for use in performing a prostatectomy procedure. The method 200 begins with step 210, wherein a dominant lesion in the prostate gland is identified, and its location is determined. The dominant lesion 12 and its location may be identified in a number of ways known in the art. One way is through use of one or more known imaging techniques or modalities (e.g., magnetic resonance imaging (MRI), ultrasound, and/or another suitable imaging technique or modality known in the art). Another way is through use of a transrectal biopsy procedure or a transperineal prostate biopsy procedure, both of which are well known in the art. While particular ways of identifying the dominant lesion and its location have been identified, it will be appreciated the present disclosure is not intended to be limited to any particular way of doing so, but rather any suitable way now known or later developed may be used.

Following the identification of the dominant lesion and the determination of its location, the method 200 proceeds to step 220. In step 220, one or more cuts are made outside the perimeter of the prostate capsule in a region of the capsule where the dominant lesion is located, and inside the perimeter of the prostate capsule in a region of the prostate capsule where the dominant lesion is not located. As an example, the template 10 depicted in FIG. 1 is representative of an example of one or more cuts that is/are made outside the perimeter of the capsule where the dominant lesion 12 is located, and inside the perimeter of the prostate capsule in a region of the prostate capsule where the dominant lesion 12 is not located. In one embodiment, the cut(s) may be made outside the perimeter of the prostate capsule in a region of the prostate that is on the same side of an anterior-posterior midline of the prostate as the dominant lesion, and the cut(s) may be made inside the perimeter of the prostate capsule in a region of the prostate that is on the opposite side of the anterior-posterior midline from the dominant lesion. For example, in FIG. 1, the template 10 is drawn outside the perimeter of the prostate capsule in a region that is located on the same side as the dominant lesion 12 and is drawn inside the perimeter of the prostate capsule in a region that is located on the opposite side as the dominant lesion 12. Also, in an embodiment, the cut(s) may be made so that the seminal vesicles and/or vas deferens that are on the same side of the anterior-posterior midline 20 as the dominant lesion are cut out thereby allowing them to be subsequently removed. Additionally, in an embodiment, the cut(s) may be made so that the seminal vesicles and/or vas deferens that are on the opposite side of the anterior-posterior midline 20 from the dominant lesion are not cut out thereby allowing them to be preserved.

In an embodiment wherein one or more cuts are made inside the perimeter of the capsule, the cuts may be made a predetermined distance inward of the outer periphery or outer surface of the capsule. In an embodiment, this distance may be 1-10 mm from the outer periphery and, in a particular embodiment, this distance may be 4-8 mm from the outer periphery, although lesser or greater distances may certainly be used instead. In one embodiment, the cuts may be made using robotic cutting tools or implements, which may include, for example and without limitation, cautery tools or implements and/or sharpened tools or implements.

Following the cut(s) performed in step 220, the method 200 proceeds to step 230 in which the prostate gland and a first portion of the prostate capsule is removed from the patient's body, and a second portion of the prostate capsule is preserved in the patient's body and left at least substantially intact (e.g., the nerves attached thereto are left intact). In at least one embodiment, the first portion of the capsule that is removed is a portion of the capsule located in the region in which the cut(s) made in step 220 was/were made outside of the perimeter of the capsule. Thus, in this embodiment, the first portion is inside of the cut(s) made in step 220 (i.e., the portion of the prostate capsule that is inside template 10 (FIG. 1)). In at least one embodiment, the second portion of the prostate capsule that is preserved is a portion of the capsule located in the region in which the cut(s) made in step 220 was/were made inside of the perimeter of the capsule. Thus, in this embodiment, the second portion is outside the cut(s) made in step 220 (i.e., the portion of the prostate capsule that is outside template 10 (FIG. 1)). In other words, the first portion of the prostate capsule that is removed corresponds a region of the prostate gland in which the dominant lesion is located, and the second portion of the prostate capsule that is preserved corresponds to a region of the prostate gland in which the dominant lesion is not located.

The region of the prostate gland in which the dominant lesion is located may be defined in a number of ways. For example, this region may include only the portion of the prostate gland corresponding to the location of the dominant lesion. Alternatively, this region may include the portion of the prostate gland corresponding to the location of the dominant lesion as well as adjacent portions of the gland extending a certain distance beyond the periphery of the dominant lesion in one or more radial directions. In one embodiment, the first portion may include the parts of the prostate capsule that are located on the same side of the anterior-posterior midline of the prostate as the dominant lesion. In at least some embodiments, the cut(s) may be made so as to cut out (and subsequently remove) a seminal vesicle and/or vas deferens that are on the same side of the anterior-posterior midline as the dominant lesion. As shown in FIG. 1, the second portion of the prostate capsule corresponds to the remnant capsular tissue 16, which, in an embodiment, is located on the opposite side of the anterior-posterior midline of the prostate from the dominant lesion.

Following the removal of the prostate gland and the first portion of the prostate capsule in step 230, the method moves to step 240 in which one or more samples are collected or removed from the preserved second portion of the capsule (i.e., the remnant capsular tissue) for purposes of testing for the presence of cancer cells in the remnant capsular tissue. In one embodiment, the second portion may include the portion of the prostate capsule that is on the side of the anterior-posterior midline of the prostate that is opposite from the dominant lesion—that is, when the dominant lesion is on a first side of the anterior-posterior midline, the second portion may include the prostate capsule that is on the second side of the anterior-posterior midline. In at least some embodiments, the cut(s) may be made so as to preserve a seminal vesicle and/or vas deferens that are on this second side of the anterior-posterior midline. As briefly described above, any number of samples may be collected during this step of the procedure to allow for a thorough examination of the remnant capsular tissue. For example, in one embodiment, 1-15 real-time biopsies are performed, and in another embodiment, 8-10 biopsies are performed. It will be appreciated, however, that the present disclosure is not intended to be limited to any particular number of biopsies.

The samples may be collected in step 240 using any number of techniques known in the art, including, for example and without limitation, an image-guided (e.g., ultrasound, MRI, etc.) transperineal or transrectal technique, both of which are known in the art. For example, in an embodiment wherein a transperineal technique is performed, the samples may be collected using the Trinity® Perine system commercially available from KOELIS SA located in La Tronche, France. In addition to being configured to collect samples of capsular tissue, this system may also be used to generate a three-dimensional (3-D) model of the prostate from image data corresponding to the prostate, and to map onto the 3-D model each sample site to allow the physician to see where samples are collected from.

Following step 240, method 200 may proceed to a step 250 in which the sample(s) collected in step 240 are tested for the presence of cancer cells and a determination is made as to whether cancer cells are present in one or more of the collected samples, and thus, whether cancer cells are present in the second portion of the capsule (i.e., the remnant capsular tissue). The sample(s) may be tested using known techniques for detecting the presence of cancer cells, and both the sample collection and testing may be done intra-operatively while the patient is still in the operating room and during the same procedure in which steps 220 and 230 were performed.

As illustrated in FIG. 2, when cancer cells are detected in one or more samples collected from the second portion of the capsule, the method 200 may proceed to step 260 described below. Otherwise, the method 200 may proceed to step 270, which comprises preserving or leaving the second portion of the capsule at least substantially intact in the patient's body.

In step 260, at least part of the second portion of the capsule is removed. In one embodiment, the entire second portion is removed. In other embodiments, however, one or more parts or areas of the second portion may be removed while one or more other parts or areas of the second portion may be preserved. In an embodiment, the part(s) of the second portion to be removed may be removed by cutting outside the perimeter of the second portion of the prostate capsule as described elsewhere above, or by using any other suitable technique.

More specifically, if it is determined from the testing of the samples collected in step 240 that cancer cells are not present in one or more parts of the second portion, one or more of those one or more parts may be preserved and left at least substantially intact (i.e., not removed) while parts determined to contain cancer cells, or, in an embodiment, parts of the second portion within a certain threshold distance of the location of a sample determined to contain cancer cells, may be removed. In an embodiment, the certain threshold distance may be a predetermined distance that is defined prior to the performance of the procedure, or alternatively may be a distance defined by a physician during the performance of the procedure.

In an embodiment wherein at least one part of the second portion is removed and at least one other part of the second portion is preserved in step 260, method 200 may comprise repeating steps 240-270 for the preserved part(s) of the second portion so as to provide an iterative means for examining the remnant capsular tissue.

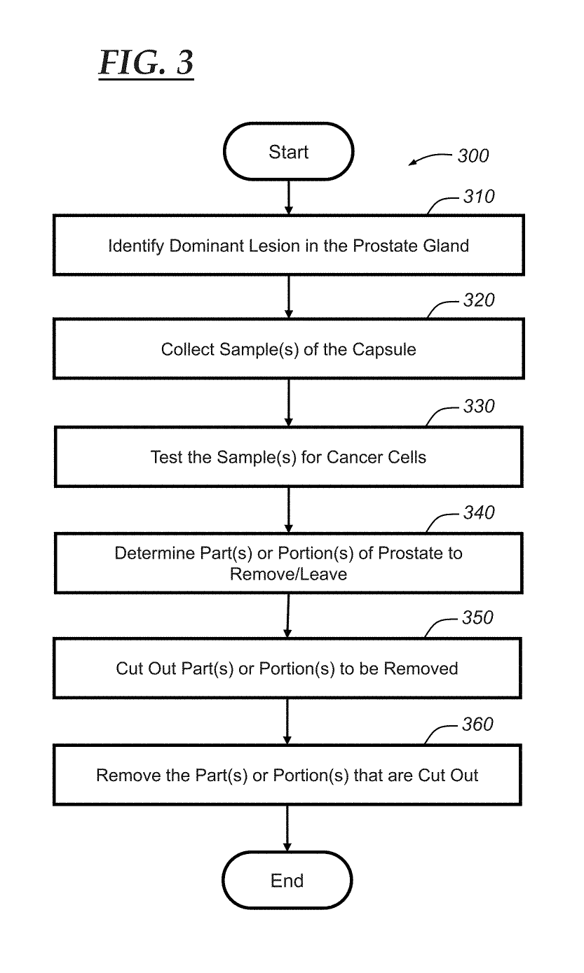

With reference to FIG. 3, there is shown a second embodiment of a method (method 300) for use in performing a prostatectomy procedure. The method 300 begins with step 310, wherein a dominant lesion in the prostate gland is identified, and its location within the prostate gland determined. Like step 210 of method 200 described above, the dominant lesion and its location may be identified or determined in a number of ways known in the art. One way is through use of one or more known imaging techniques or modalities (e.g., MRI, ultrasound, and/or another suitable imaging modality). Another way is through use of a transrectal biopsy procedure or a transperineal biopsy procedure, both of which are well known in the art. While particular ways of identifying the dominant lesion and its location have been identified, it will be appreciated that the present disclosure is not intended to be limited to any particular way of doing so, but rather any suitable way now known or later developed may be used.

In an embodiment, step 310 (or another step of method 300) may also optionally comprise identifying or defining a first portion of the prostate capsule that corresponds to the dominant lesion and that is/are intended to be removed from the patient's body during a later step of method 300. In such an embodiment, the first portion of the prostate capsule may be defined to correspond only to a region of the prostate gland where the dominant lesion is located (e.g., the portion of the capsule located on the same side of the anterior-posterior midline of the prostate as the dominant lesion). Alternatively, the first portion may be defined to correspond to both the region of the prostate gland where the dominant lesion is located, as well as a portion of the prostate gland within a certain distance of the dominant lesion in one or more radial directions from the lesion. In the latter instance, the certain distance may be a predetermined distance that is defined prior to the performance of the procedure or alternatively may be a distance defined by a physician during the performance of the procedure.

Following step 310, the method 300 continues to step 320 in which one or more samples are collected or removed from the prostate capsule and tested for the presence of cancer cells. In an embodiment such as that described above wherein a first portion of the prostate capsule is defined or identified as a portion of the capsule to be removed, the samples collected in step 320 may be collected from parts or portions of the prostate capsule other than the first portion of the capsule. That or those part(s) or portion(s) may comprise one or more portions of the capsule that a physician would like to preserve if possible (i.e., not remove but rather leave at least substantially intact). In another embodiment, rather than just collecting samples from a portion of the capsule intended to be preserved, step 320 may comprise collecting samples from any portion or location of the prostate capsule that is accessible for sample collection.

In any event, step 320 is similar to step 240 of the method 200 (FIG. 2) discussed above, except that in this embodiment, the sampling step is carried out prior to a cutting step (step 350 in method 300 described below) and removal step (step 360 in method 300 described below). Step 320 may be carried out in a manner similar to that of step 240 (FIG. 2).

In an embodiment, whether the sample(s) collected in step 320 are from a particular defined or targeted portion of the capsule (e.g., a portion intended to be preserved) or not, as a part of this step, potential collection sites may be identified based on a location of the dominant lesion or on one or more anticipated cut lines. An anticipated cut line may be a line extending along the prostate either inside or outside of the perimeter of the capsule and along which one or more cuts are intended to be made so that the prostate gland (or at least a portion of the gland containing the dominant lesion) and at least a portion of the prostate capsule may be removed. The anticipated cut line may be determined based on the location of the dominant lesion. Thus, based on the location of the dominant lesion, one or more potential collection sites may be identified. As described above, in an embodiment, the potential collection site(s) may be located in one or more portions of the capsule intended to be preserved.

As with step 240 of method 200 described above, any number of samples may be collected during step 320 to allow for a thorough examination of the prostate capsule or at least a portion thereof. Accordingly, it will be appreciated that the present disclosure is not intended to be limited to any particular number of samples. Further, the samples may be collected in step 320 in the same or similar manner as that described above in connection with step 240 of method 200. As such, unless otherwise indicated herein, the description of step 240 applies to step 320 with equal weight and will not be repeated but rather is incorporated here by reference in its entirety.

Following step 320, method 300 may proceed to a step 330 in which the sample(s) collected in step 320 are tested for the presence of cancer cells and a determination is made as to whether or not cancer cells are present in one or more of the collected samples. If it is determined that cancer cells are present in one or more samples, the location(s) of that or those sample(s) is identified, and/or if it is determined that cancer cells are not present, the location(s) of that or those sample(s) may be identified.

Based at least in part on the testing performed in step 330, in a step 340 a decision or determination is made by a physician as to whether the entire prostate (i.e., the gland and the entirety of the capsule) or less than the entire prostate is to be removed, and the part(s) or portion(s) of the capsule to actually be removed and/or the part(s) or portion(s) of the capsule to be preserved (if any) are identified. More specifically, when no cancer cells are detected in any of the samples tested in step 330 the physician may determine that at least a portion of the prostate capsule may be preserved and not removed. In an embodiment, the particular portion(s) that is/are to be preserved may be up to the physician's judgment, but may include, for example, the entire capsule or one or more portions of the capsule located at a particular location relative to the dominant lesion (e.g., on the other side of the anterior-posterior midline from the dominant lesion) or at least a certain distance away from the dominant lesion in the prostate gland. In the latter instance, the certain distance may be a predetermined distance that is defined prior to the performance of the procedure, or alternatively may be a distance defined by a physician during the performance of the procedure.

On the other hand, when cancer cells are detected in one or more of the samples tested in step 330, the physician may decide to remove all or part of the capsule. For example, in one embodiment, the physician may decide to remove the entire prostate gland and the entire prostate capsule, effectively resulting in a radical removal of the prostate. Alternatively, the physician may decide to remove those parts of the capsule where cancer cells were detected and/or that are within a certain distance from the parts of the capsule where cancer was detected, but also preserve one or more other parts of the capsule where cancer cells were not detected, or at least one or more parts where cancer is not detected and that is/are also a certain distance away from parts of the capsule where cancer was detected. The certain distance(s) may be a predetermined distance that is defined prior to the performance of the procedure, or alternatively may be a distance defined by a physician during the performance of the procedure.

Once it is determined in step 340 which part(s) or portion(s) of the prostate capsule are to be removed and which part(s) or portion(s) are to be preserved (if any), the method 300 moves to a step 350, of cutting out only the parts or portions of the prostate capsule to be removed and not cutting out the parts or portions of the capsule that is/are to be preserved. The cut(s) may be made using any one or more of those techniques or tools discussed above with respect to step 220 of method 200. In an embodiment, this comprises making one or more cuts outside of the perimeter of the portions or parts of the prostate capsule to be removed, and inside the perimeter of the portions or parts of the prostate capsule to be preserved. More particularly, if it is determined in step 340 that the entire prostate capsule is to be removed, step 350 comprises making one or more cuts outside the entire perimeter of the prostate capsule.

On the other hand, if it is determined that one or more portions of the capsule is/are to be preserved, step 350 comprises making one or more cuts outside of the perimeter of the prostate capsule for the parts or portions of the prostate gland and capsule being removed, and inside the perimeter of the parts or portions of the prostate capsule being preserved. In an embodiment wherein one or more cuts are made inside the perimeter of the capsule, the cuts may be made a predetermined distance inward of the outer periphery or surface of the capsule. In an embodiment, this distance may be 1-10 mm from the outer periphery, and, in a particular embodiment, this distance may be 4-8 mm from the outer periphery, though lesser or greater distances may certainly be used instead.

Once the necessary cuts are may in step 350, the method 300 may move to a step 360 of removing from the patient's body the portions or parts of the prostate that have been cut and that are to be removed. As with the embodiment described above and shown in FIG. 2, in at least some embodiments of method 300, the cutting step 350 and removing step 360 may also include cutting out and removing one or more seminal vesicles and vas deferens (e.g., the seminal vesicle and vas deferens on the same side of the anterior-posterior midline as the dominant lesion. In other embodiments, however, one or more seminal vesicles and vas deferens may be preserved (e.g., the seminal vesicle and vas deferens on the opposite side of the midline from the dominant lesion).

In an embodiment wherein at least one or more parts or portions of the prostate capsule are preserved and not cut in step 350, method 300 may include further steps for examining the remnant capsular tissue that was preserved. These steps (not shown) may comprise collecting one or more samples from one or more of the preserved part(s) or portion(s) of the capsule, and then testing that or those samples for the presence of cancer cells. The sample(s) may be collected and tested in the same or similar manner as that described above in connection with steps 240 and 250 of method 200. As such, the description of steps 240 and 250 applies here with equal weight and will not be repeated but rather is incorporated here by reference in its entirety.

If no cancer cells are detected in any of the collected samples, the method may comprise preserving the entirety of the part(s) or portion(s) of the preserved remnant capsular tissue in the patient's body. If, on the other hand, cancer cells are detected in one or more of the collected samples, the method 300 may proceed to a step in which at least part of the remnant capsular tissue is removed. This step is substantially the same as step 260 of method 200, and as such, the description of steps 260 applies here with equal weight and will not be repeated but rather is incorporated here by reference in its entirety. Following the removal of at least part of the remnant tissue, the steps of collecting, testing, and removing/preserving tissue may be repeated one or more times.

One benefit of the method described above is that there is a reasonable likelihood that preserving a portion of the prostate capsule will result in greater nerve protection and lessening the chance of nerve damage than if the entire prostate capsule was to be removed. This is because less of the nerves are removed or peeled back. As a result, there is a greater likelihood for better recovery or maintenance of urinary and/or erectile function. In experimental procedures performed to validate the efficacy of the method described above, it was found that 95% of patients regained urinary control and 90% achieved satisfactory erections within four (4) weeks of the procedure. By way of comparison, at three (3) to four (4) months after a complete prostatectomy, 95% of patients are continent, 15% had or have natural erections, and another 30% have erections sufficient for intercourse with injections and/or mechanical assistance.

Further, most patients having a complete prostatectomy have an undetectable prostate-specific antigen (PSA) value post-procedure. Because a portion of the prostate capsule is being left intact in the procedure described above, a thin layer of prostate cells remains, and thus, cells are left behind that will continue to produce PSA. For the patients on whom the experimental procedures were performed, while the median PSA value was below 1.0 ng/mL one (1) to twelve (12) months post-surgery, it was nevertheless detectable.

It is to be understood that the foregoing is a description of one or more embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A method for use in performing a prostatectomy procedure on a patient, comprising:
identifying a dominant lesion in a prostate gland, wherein the prostate gland is contained within a prostate capsule;
cutting outside the perimeter of a first portion of the prostate capsule corresponding to a region of the prostate gland in which the dominant lesion is located, and inside the perimeter of a second portion of the prostate capsule corresponding to a region of the prostate gland in which the dominant lesion is not located; and
removing the first portion of the prostate capsule from the patient's body and preserving the second portion of the prostate capsule in the patient's body.

2. The method of claim 1, wherein the method further comprises collecting one or more samples of the second portion of the prostate capsule and testing the sample(s) for cancer cells.

3. The method of claim 2, wherein when cancer cells are detected based on the testing of the sample(s), the method further comprises removing at least a part of the second portion of the prostate capsule.

4. The method of claim 3, further comprising cutting outside of the perimeter of the part(s) of the second portion of the prostate capsule to be removed.

5. The method of claim 3, wherein the step of removing at least a part of the second portion of the prostate capsule comprises removing a first part of the second portion of the prostate capsule corresponding to a location at which cancer cells were detected and preserving a second part of the second portion of the prostate capsule corresponding to a location at which cancer cells were not detected.

6. The method of claim 5, further comprising cutting outside the perimeter of the first part of the second portion of the prostate capsule.

7. The method of claim 3, wherein the step of removing at least a part of the second portion of the prostate capsule comprises removing the entire second portion of the prostate capsule.

8. The method of claim 7, further comprising cutting outside the perimeter of the second portion of the prostate capsule.

9. A method for use in performing a prostatectomy procedure on a patient, comprising:
identifying a dominant lesion in a prostate gland, wherein the prostate gland is contained within a prostate capsule;
collecting one or more samples of the prostate capsule and testing the sample(s) for cancer cells;
identifying, based on the testing of the sample(s), a first portion of the prostate capsule to remove from the patient's body, and one or more other portions of the prostate capsule to preserve;
cutting outside the perimeter of the first portion of the prostate capsule to be removed, and inside the perimeter of the one or more other portions of the prostate capsule to be preserved; and
removing from the patient's body the first portion of the prostate capsule and preserving the one or more other portions of the prostate capsule in the patient's body.

10. The method of claim 9, wherein following the removal of the first portion of the prostate capsule, the method further comprises collecting one or more samples from the one or more preserved portions of the prostate capsule and testing the sample(s) for cancer cells.

11. The method of claim 10, wherein when cancer cells are detected in one of the one or more preserved portions of the prostate capsule, the method further comprises removing at least a part of that preserved portion of the prostate capsule.

12. The method of claim 11, further comprising preserving another part of that preserved portion of the prostate capsule.

13. The method of claim 10, wherein when cancer cells are detected in a first one of the one or more preserved portions of the prostate capsule but not in a second one of the one or more preserved portions, removing at least a part of the first one of the one or more preserved portions and preserving at least a part of the second one of the one or more preserved portions.

14. The method of claim 10, wherein when cancer cells are detected in at least one of the one or more preserved portions, the method further comprises removing all of the one or more preserved portions from the patient's body.

15. A method for use in performing a prostatectomy procedure on a patient, comprising:
identifying a dominant lesion in a prostate gland, wherein the prostate gland is contained within a prostate capsule;
identifying a first portion of the prostate capsule to remove from the patient's body and a second portion of the prostate capsule to preserve; and
removing the first portion of the prostate capsule from the patient's body and preserving the second portion of the prostate capsule in the patient's body.

16. The method of claim 15, further comprising cutting outside the perimeter of the first portion of the prostate capsule to be removed, and inside the perimeter of the second portion of the prostate capsule to be preserved.
